(19) United States
(12) Patent Application Publication
(10) Pub. No.: US 2014/0099692 A1
(43) Pub. Date: Apr. 10, 2014

(54) INTEGRATED METHOD OF PRODUCING CALCITE AND BIOMASS USING CYANOBACTERIA FOR ENERGY VALORIZATION AND MINERAL SEQUESTRATION OF CO2

(71) Applicant: IFP Energies Nouvelles, Rueil-Malmaison Cedex (FR)

(72) Inventors: Denis Blanchet, Le Vesinet (FR); Frank Haeseler, La Garenne Colombes (FR); Lun Li, Rueil-Malmaison (FR); Gilles Dromart, Sainte Foy Les Lyon (FR); Philippe Oger, Lyon (FR)

(21) Appl. No.: 14/105,625

(22) Filed: Dec. 13, 2013

Related U.S. Application Data
(63) Continuation of application No. 13/881,734.

(30) Foreign Application Priority Data
Oct. 28, 2010 (FR) 10/04.248

(51) Int. Cl. C12P 3/00; C05F 17/00; C12P 5/02

(52) U.S. Cl. CPC C12P 3/00; C12P 5/023; C05F 17/0054
USPC 435/167; 435/168

(57) ABSTRACT

The invention relates to a CO₂ biological capture method comprising the implementation of a photosynthesis reaction by cyanobacteria on the hydrogen carbonate ions of a carbonate system comprising calcium, allowing biomass and calcite (CaCO₃) to be produced.

The invention allows energy valorization and mineral sequestration of CO₂.

### CROSS-REFERENCE TO RELATED APPLICATION

This application is a continuation application of U.S. application Ser. No. 13/881,734, filed Apr. 26, 2013, which is a national stage application under 35 U.S.C. 371 of international application no. PCT/FR2011/000579, filed Oct. 27, 2011, the contents of each of which are incorporated herein by reference.

### FIELD OF THE INVENTION

The invention relates to the sphere of carbon dioxide (CO₂) sequestration, notably the biological capture of CO₂, using bacteria of cyanobacteria type.

The invention also relates to the sphere of energy valorization, notably the valorization of biomass.

### BACKGROUND OF THE INVENTION

At the level of the planet, phytoplankton are at the origin of primary production. They contribute, at the level of the oceans, to the consumption of CO₂ through the agency of photosynthesis on the hydrogen carbonate species.

Cyanobacteria, which are prokaryotes (unicellular organisms whose cell structure comprises no nucleus), make up one of the biological components of these phytoplankton. These cyanobacteria use, in the marine medium, the hydrogen carbonate ion HCO₃₋ as the mineral carbon species, and not the dissolved CO₂. Indeed, in sea water, the dissolved CO₂ concentration is low in relation to the hydrogen carbonate ion. The other mineral carbon species of this marine medium, carbonate, is not used by the phytoplanktonic microorganisms as a carbon source via photosynthesis.

Unicellular prokaryotic organisms of cyanobacteria type, as well as certain eukaryotic organisms, can produce through photosynthesis on the hydrogen carbonate ion not only biomass, but also calcium carbonate (referred to as calcite), provided that calcium is present in the medium and that the culture conditions are suitable.

Besides, the biomass that consists of the elements carbon, oxygen, nitrogen, hydrogen and sulfur (CONHS) represents a potential source of energy valorization.

The valorization of biomass, in particular algae biomass, can be achieved using lipids, notably for the production of fatty acid methyl esters used as biofuels, in addition for example in the petroleum distillate “diesel fuel”. This lipid valorization requires using microalgae selected for their high lipid contents. Cyanobacteria are not known to produce significant lipid amounts and they are therefore of little interest for the production of biofuels. On the other hand, some of these strains are known to use atmospheric nitrogen (N₂) as the nitrogen source for biomass synthesis.

Finally, cyanobacteria can allow to produce a valorizable biomass for hydrogen production. Cyanobacteria are in fact mentioned in the literature for their properties allowing to redirect the electron flux from the two photosynthesis stages for hydrogen production. Several paths have been identified, one of them using for example dehydrogenase of the strain, but many problems remain to be solved.

Finally, biomass valorization through anaerobic digestion is an interesting option. Anaerobic degradation of the algae biomass in general has been studied intensively. It is considered as a promising path that has been examined both on the entire biomass and on the residual fraction in a development scheme for lipids from biomass. When the lipids do not exceed 40% of the composition of the algae biomass of the microorganisms selected for biofuel production, it appears that direct anaerobic digestion of all of the biomass is more favourable as regards energy than the succession of lipid extraction stages (for biofuels), then of residual biomass anaerobic digestion stages.

Furthermore, implementing this valorization through anaerobic digestion of the algae biomass poses many problems. The main barriers have been identified. The biodegradability of the algae biomass depends on the composition of the wall-membrane network of these microalgae. Besides, the high nitrogen content leads to the discharge, into the digester, of high ammonia concentrations that eventually inhibit the reaction. Finally, the presence of sodium in the case of biomass produced on marine media (use of marine strains) can also affect the performances of the anaerobic digestion process.

Due to their low lipid content, cyanobacteria are not of interest for the production of biofuels, but they can be used for the production of biomass through growth in an aqueous medium in a carbonate system and for the subsequent anaerobic digestion of this cultured biomass on hydrogen carbonate. Laboratory studies show that, at the end of the reaction, when the hydrogen carbonate ion is totally consumed, a lysis process occurs rapidly, which positions the cyanobacterial biomass in a more favourable digestibility situation than the biomass from microalgae.

Furthermore, another advantage of this biomass is that it can be used directly as a fertilizer, considering its nitrogen content.

The method according to the invention uses cyanobacteria culture and it allows to solve all the aforementioned problems related to biomass valorization insofar as it provides both corrections to the implementation of biomass production processes leading to manage the distribution, within the algae biomass, among the proteins, the lipids and the sugars, as well as enzymatic and physical pretreatments intended to improve the “digestibility” or the “biodegradability” of these structures present in the wall and the membrane. Thus, surprisingly enough, it appears that growing cyanobacteria under optimized conditions on a carbonate system in the presence of calcium allows, through consumption of the hydrogen carbonate ion, to provide two products, one in form of a readily valorizable cyanobacterial biomass and the second in form of calcite (calcium carbonate CaCO₃) that makes up a carbon trap. Valorization of these two products, biomass and calcium carbonate, can be performed in different ways.

### OBJECTS OF THE INVENTION

The invention relates to a CO₂ biological capture method through biomass and calcite production using cyanobacteria, wherein cyanobacteria culture is carried out on a carbonate system (comprising carbonate ions CO₃²⁻ and hydrogen carbonate ions HCO₃₋), in the presence of calcium, with pH regulation through controlled CO₂ injection.

The invention also relates to the use of the biomass obtained with the method as a fertilizer or for energy purposes.

### SUMMARY OF THE INVENTION

The present invention relates to an integrated CO₂ biological capture method using cyanobacteria, comprising the following stages:

a) growing cyanobacteria in the presence of calcium in an aqueous medium comprising carbonate ions and hydrogen carbonate ions ;

b) achieving photosynthesis by the cyanobacteria on the hydrogen carbonate ions so as to produce a cyanobacterial biomass consisting of the elements carbon, oxygen, nitrogen, hydrogen and sulfur, and to cause precipitation of calcium carbonate (CaCO₃) ;

c) supplying inorganic carbon by injecting CO₂ in order to regulate the pH during the photosynthesis reaction.

The CO₂ can be atmospheric and the cyanobacteria can be grown in at least one open reactor.

At least part of the CO₂ injected can come from industrial fumes/discharges.

Stages a), b) and c) can be repeated until exhaustion of the calcium in the medium.

The pH can be regulated to a value ranging between 9 and 10.

The culture medium can be a synthetic aqueous medium containing calcium and a carbonate system, or a marine medium.

Nitrogen can be supplied to the medium in form of nitrate ions.

The nitrate ions can be calcium nitrate.

Cyanobacteria can be grown under continuous or semi-continuous conditions, or batchwise.

A stage of anaerobic digestion of the cyanobacterial biomass can be carried out in order to convert the biomass to methane.

The anaerobic digestion stage can be carried out by injecting the biomass into an anaerobic digester during the nocturnal phase.

The biomass anaerobic digestion stage can be carried out directly after the production stage.

The biomass can be separated from the medium water for thermal valorization purposes.

The biomass can be valorized by direct combustion and heat recovery, or by pyrolysis in order to generate an oil.

The biomass obtained can be used as an energy source.

The biomass obtained can be used as a fertilizer.

The method can be used for calcite production (CaCO₃) and for CO₂ mineral sequestration.

### BRIEF DESCRIPTION OF THE FIGURES

FIGS. 1 to 8 illustrate the various aspects of the invention by way of non limitative example.

FIG. 1 shows the evolution of the biomass concentrations and of the following species: calcium, CO₃²⁻, HCO₃₋, as well as the evolution of the pH value during the hydrogen carbonate assimilation by the cyanobacteria, batchwise, in the absence of calcium, and without inorganic carbon supply during culture,

FIGS. 2a and 2b show the evolution of the biomass concentrations and of the following species: calcium, CO₃²⁻, HCO₃₋, as well as the evolution of the pH value during the hydrogen carbonate assimilation by the cyanobacteria, batchwise, in the presence of calcium (FIG. 2a: the hydrogen carbonate is present in large excess in relation to the calcium ; FIG. 2b: the calcium is present in large excess in relation to the hydrogen carbonate). In this case also, culture is achieved without inorganic carbon supply,

FIG. 3 shows the hydrogen carbonate assimilation for an initial calcium, CO₃²⁻, HCO₃₋ composition representative of that of sea water,

FIG. 4 highlights the shuttle role played by the carbonate/hydrogen carbonate pair in the marine medium,

FIG. 5 shows the evolution of the carbonate and hydrogen carbonate concentrations as a function of the carbon synthesized in the biomass (comparison between the experimental values and the theoretical values of the hydrogen carbonate and carbonate concentrations),

FIG. 6 shows the evolution of the CO₂ consumptions as a function of the carbon synthesized in the biomass (comparison between the experimental values and the theoretical values of CO₂ injected),

FIG. 7 shows the data of tests A and B,

FIG. 8 illustrates a biomass and calcite production device integrated in an energy valorization method.

### DETAILED DESCRIPTION

The method according to the invention is an integrated CO₂ biological capture method using cyanobacteria and comprising the following stages:
growing cyanobacteria in an open or closed aqueous medium, in the presence of calcium on a carbonate system (comprising hydrogen carbonate ions and carbonate ions),
production of a cyanobacterial biomass consisting of the elements carbon, oxygen, nitrogen, hydrogen and sulfur by photosynthesis on the hydrogen carbonate ion,
associated calcium carbonate (or calcite or CaCO₃) precipitation,
culture pH regulation through controlled CO₂ injection according to the biomass production and to the precipitation events.

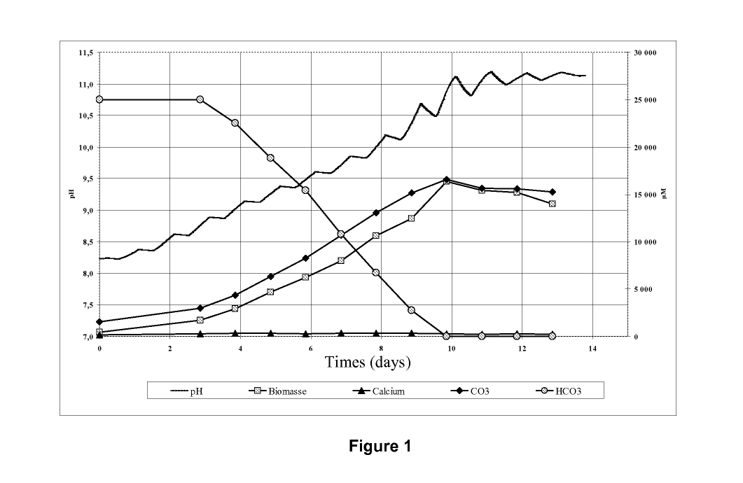

In order to allow to understand the phenomena involved in the method according to the invention, the implementation of a cyanobacteria culture on an aqueous system comprising hydrogen carbonate ions and possibly carbonate ions under various conditions, as well as the fluxes of the different species, are described in the paragraphs below (relative to FIGS. 1 to 8).

Hydrogen Carbonate Assimilation by the Cyanobacteria in Batch Culture and Calcium Carbonate Production

In the Absence of Calcium:

FIG. 1 shows the evolution of the main parameters (pH, biomass, calcium, CO₃²⁻ and HCO₃₋ concentration) during the assimilation of hydrogen carbonate by a cyanobacteria culture, in batch culture, i.e. in a closed medium, i.e. without additional inorganic carbon supply and in the absence of calcium. Assimilation of the hydrogen carbonate with biomass and carbonate production is observed. The pH profile shows an alkalinization of the medium through the evolution of the carbonate system (hydrogen carbonate concentration drop and carbonate concentration increase). The pH profile also highlights the day and night phase alternations, with a photosynthesis activity during the day phase and a respiration activity during the night phase.

A balance shows that, for two hydrogen carbonate ions consumed, one carbon coming from the hydrogen carbonate is distributed in the biomass and another carbon coming from the hydrogen carbonate is converted to carbonate.

In the Presence of Calcium:

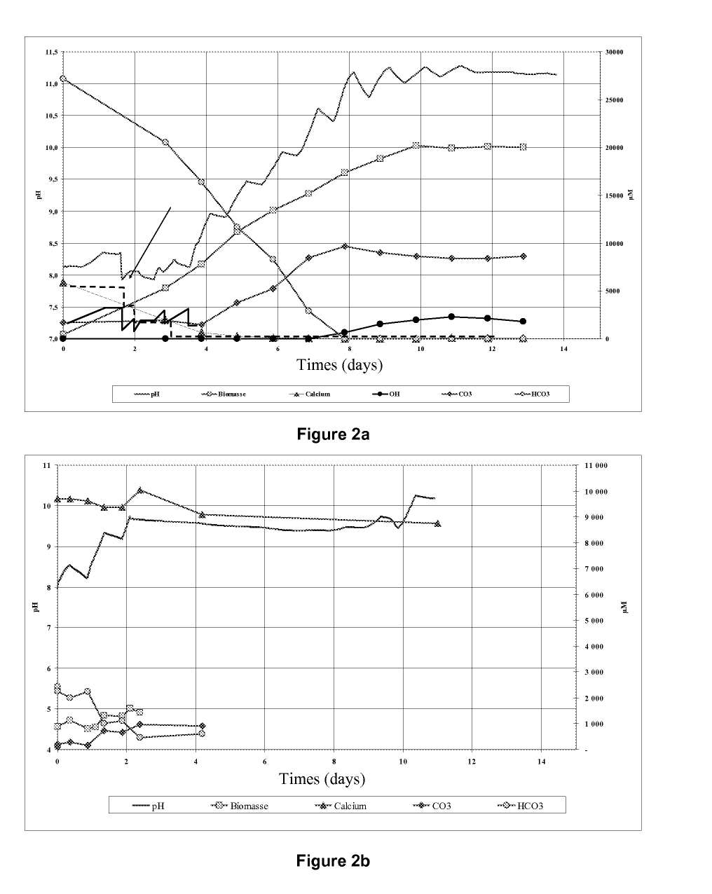

FIGS. 2a and 2b show the evolution of these parameters when the hydrogen carbonate assimilation is conducted in the presence of calcium.

In the first case (FIG. 2a), the hydrogen carbonate concentration at the beginning of the test is in large excess in relation to the calcium concentration. At the beginning of this batch test (2a) in the presence of calcium, the carbonate concentration increases (hydrogen carbonate consumption) up to a concentration where fast modification of the pH profile occurs (pH drop). This pH drop is associated with a decrease in both the carbonate and the calcium concentrations (the respective concentration decrease amplitudes are in a ratio of 1/1) that indicates a calcium carbonate precipitation (CaCO₃). The hydrogen carbonate consumption rate is hardly altered by this calcium carbonate precipitation event.

In this test (2a), if one considers the final balance at the end of the total hydrogen carbonate consumption, all of the calcium present is sequestered in form of CaCO₃. Hydrogen carbonate remains, which is then strictly assimilated in biomass and in carbonate (and no longer in CaCO₃), as in the instance of growth in the absence of calcium presented in the previous paragraph in connection with FIG. 1.

In the second test (FIG. 2b), the calcium is initially in large excess in relation to the hydrogen carbonate. During the course of the hydrogen carbonate ion assimilation reaction, fast modification of the pH profile is observed (pH drop). This pH drop is associated with a decrease in the carbonate and calcium concentrations (the respective concentration decrease amplitudes are in a ratio of 1/1) that indicates a calcium carbonate precipitation. The hydrogen carbonate consumption rate is hardly altered by this calcium carbonate precipitation event. At the end of the test, the hydrogen carbonate is totally consumed and a residual calcium concentration is observed.

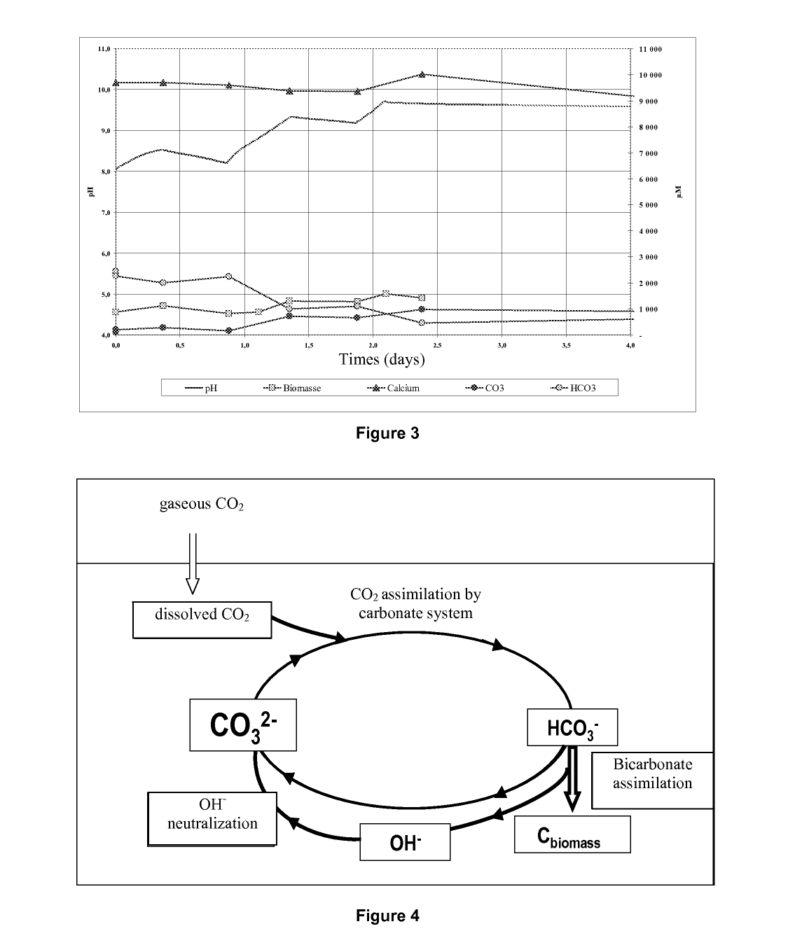

These two tests (FIGS. 2a-2b and FIG. 3) and the test without calcium (FIG. 1) show that the biomass production is totally independent of the calcium carbonate production. On the other hand, calcium carbonate production can be achieved only in the presence of calcium and of a carbonate production linked with the photosynthesis activity. The production of calcium carbonate therefore requires a photosynthesis activity on the hydrogen carbonate ion to produce the necessary carbonate ion.

Under excess hydrogen carbonate conditions for example, the carbon and calcium balances at the end of the total hydrogen carbonate consumption show that the calcium is completely engaged in calcium carbonate. The carbonate produced by photosynthetic assimilation of all of the hydrogen carbonate represents half of the carbon coming from the hydrogen carbonate and it is distributed among the carbonate ion and the calcium carbonate. Residual carbonate remains.

Under excess calcium conditions, the carbon and calcium balances at the end of the total hydrogen carbonate consumption show that the calcium is partly engaged in calcium carbonate, but that it also exists in ionic form (free form not engaged in calcium carbonate). The carbonate produced by photosynthetic assimilation of all of the hydrogen carbonate represents half of the carbon coming from the hydrogen carbonate and it is fully found in the sum C_{calcium carbonate} and C_{carbonate}. Residual calcium thus remains.

Under optimum conditions, if a minimum concentration above a threshold value is provided both for the hydrogen carbonate and the calcium, and besides under stoichiometric conditions equal to ½ (calcium/hydrogen carbonate), an optimum calcite precipitation is obtained by stoichiometric association of the calcium ion with the carbonate ion, with a total consumption of the hydrogen carbonate, a zero residual carbonate and a zero residual calcium. By way of example, for a calcium concentration of 10 mM, a minimum carbonate concentration of 1.1 mM is required to induce a precipitation.

A hydrogen carbonate source usable in large amounts is sea water. In sea water, the hydrogen carbonate ion predominates in relation to the dissolved CO₂. The respective concentrations of the three inorganic carbon species present in the carbonate system of sea water are as follows: HCO₃₋=1818 μmoles.kg⁻¹ water; CO₃_{^{2−}}=272 μmoles.kg⁻¹ water; dissolved CO₂: 10.4 μmoles.kg⁻¹ water, for a pH value of 8.1. In general, the calcium concentration in sea water is 10.3 mM and it is therefore much higher than that of the two carbonate species, the hydrogen carbonate ion (of the order of 5.6 times as high) and the carbonate ion (of the order of 38 times as high).

The current composition of sea water does not enable to produce calcium carbonate by spontaneous chemical precipitation. Furthermore, this chemical composition of sea water does not enable to produce calcium carbonate under batch conditions in the presence of a cyanobacteria culture, without additional inorganic carbon supply. This batchwise operation situation however allows the carbonate ions to increase, considering the conversion of the hydrogen carbonate (1818 μM) to carbonate, which leads to a theoretical final carbonate concentration of 272+1818/2=1181 μM, to be compared with the 10.3 mM calcium concentration. A test (FIG. 3) carried out under calcium, carbonate and hydrogen carbonate concentration conditions very close to those of the composition of sea water, with 180 μM carbonate, 2261 μM hydrogen carbonate and 10.3 mM calcium, allows to obtain a final concentration close to the theoretical carbonate concentration through the photosynthetic use of the hydrogen carbonate of 1130 μM+180 μM=1300 μM. Under such conditions, no calcium carbonate precipitation is observed in the batch culture. On the one hand, the pH profile shows no pH drop and, on the other hand, the carbonate concentration obtained shows that the carbonate has not been engaged, even partly, in a CaCO₃ production. Besides, the calcium content of the medium remains constant.

In conclusion, it is not possible to achieve, with a batch cyanobacteria culture (finite volume) and without supplying additional inorganic carbon, a calcium carbonate precipitation from sea water, or from a culture medium under sea water conditions (HCO₃=1818 μmoles.kg⁻¹ water; CO₃_{^{2−}}=272 μmoles.kg⁻¹ water), simply through assimilation of all of the hydrogen carbonate and the associated production of carbonate (also comprising the initial concentration).

Cyanobacteria Culture on a Carbonate System with Regulated pH and CO₂ Injection. Culture Conditions in a Calcium-Free Medium

Cyanobacteria growth with a carbonate system takes place like a batch cyanobacteria culture on hydrogen carbonate, without supplying additional inorganic carbon (results shown in FIGS. 1 to 3). Under such conditions, growth in a medium with a carbonate system leads to a carbonate system imbalance (alkalinization) generated through hydrogen carbonate/carbonate ratio modification. The modification in the implementation of the cyanobacteria culture in relation to a batch hydrogen carbonate assimilation with a non-regulated pH consists in maintaining this ratio at its initial value, i.e. in keeping the pH value constant by CO₂ injection.

The pH value indicates a ratio between the concentrations of the two species carbonate and hydrogen carbonate.

This approach in the absence of calcium allows to calculate the maximum growth rates, as a function of the pH, for a given composition of the medium. It allows to specify the conversion balances of the CO₂ injected in the carbonate system to obtain a biomass carbon, to specify the nitrogen requirements of the strain, to specify the ionic form for the nitrogen supply and finally to specify the concentration evolutions of the two species within the culture medium as a function of the concentration of the biomass present.

Under such conditions, the global growth balance of the biomass according to this control mode is approximately expressed as follows:

1CO₂→≈1 Cbiomass

The diagram of FIG. 4 allows to explain the shuttle role used by the carbonate/hydrogen carbonate pair in trapping the gaseous CO₂ in the aqueous phase of the cyanobacteria culture medium, its facilitated transport to the microorganism (in form of a soluble mineral carbon=hydrogen carbonate), and the carbonate regeneration necessary for trapping the gaseous CO₂ again through assimilation of the hydrogen carbonate by the cyanobacterium (which gives 1 C_{biomass} and 1 C_{carbonate}). In this experimental approach, the two species hydrogen carbonate and carbonate are continually regenerated and only the gaseous CO₂ injected is the source of consumed mineral carbon, and it contributes alone to the production of C_{biomass}.

Under such conditions, the instantaneous biological demand (mineral carbon demand for growth) that varies with the cellular concentration increase in the reactor is met by the pH regulation-dependent CO₂ injection.

The optimum culture pH values were determined for an optimization of the biomass productivity. These optimum pH values correspond to the minimum cyanobacterial biomass doubling time. These pH values range between 9 and 10.

Furthermore, a high (optimum) pH value represents a carbonate concentration higher than the hydrogen carbonate concentration (see carbonate/hydrogen carbonate ratio>1) and it contributes to increasing the capture efficiency for the CO₂ fed into the carbonate system.

Biomass production goes together with a demand for other elements such as nitrogen and phosphorus notably, to mention only the two most important elements for biomass. The quantitatively most important element after carbon is nitrogen. The nitrogen supply in nitrate form (NO₃₋) contributes to the nitrogen assimilation in the biomass and to a hydroxyl production in the medium. The gaseous nitrogen associated with the CO₂ supply is not used by cyanobacteria at a sufficient rate to provide the growth rates required for the method.

In this cyanobacteria culture mode in a regulated carbonate system where the pH correction agent is the CO₂ injected in the culture medium, incorporation of the nitrogen in the biomass, in nitrate form, leads to the excretion of 1 OH⁻ per mole of nitrogen incorporated and therefore of y OH⁻ per mole of C incorporated in the biomass, y representing the N/C ratio (see elemental composition of the cyanobacterial biomass). This supply of y OH⁻ per mole of biomass C is going to impact the carbonate system balance and lead to the need for an additional CO₂ injection so as to maintain the Ph at the set value. This additional CO₂ injection contributes to increasing the respective concentration of the two species (hydrogen carbonate and carbonate) while maintaining the ratio between these two species, a ratio controlled by the set value regulating the pH.

It is possible to calculate, from the initial carbonate and hydrogen carbonate concentrations (this initial situation is not only identified by the concentration ratio of the two species carbonate and hydrogen carbonate, but also by the respective concentration values of these two species), the evolution of the respective concentrations of these two ionic species (hydrogen carbonate and carbonate) as a function of the C incorporated in the biomass (coming from the CO₂ injected) on the basis of the following equations:

We denote [Hydrogen carbonate]=C1 (in concentration)

we denote [Carbonate]=C2 (in concentration)

at To we denote [Hydrogen carbonate]₁₀/[Carbonate]₁₀=C1₁₀/C2₁₀

at T we denote [Hydrogen carbonate]_{T}/[Carbonate]_{T}/=C1_{T}/C2_{T}.

For a [1 Biomass Carbon] increase (expressed in concentration in the batch reactor), we have:

C1_{T1}=C1_{T0}−y+2z

C2_{T1}=C2_{T0}+y−z

We obtain z=[C1_{T0}(1+y)+C2_{T0}(2+y)]/(2C2_{T0}+C1_{T0})

with:
y=N/C, which represents a constant for a cyanobacteria strain under predetermined culture conditions without nitrogen source limitation ;
and z, which represents the additional increase in CO₂ injected (that is expressed as an injected CO₂ concentration per unit of volume of culture medium) for a concentration increase of [1 mole] biomass carbon in the culture medium. This additional CO₂ concentration can be expressed in amount by multiplying by the volume (in liter) of the reactor.

The calculated values were checked by comparison with the experimental values.

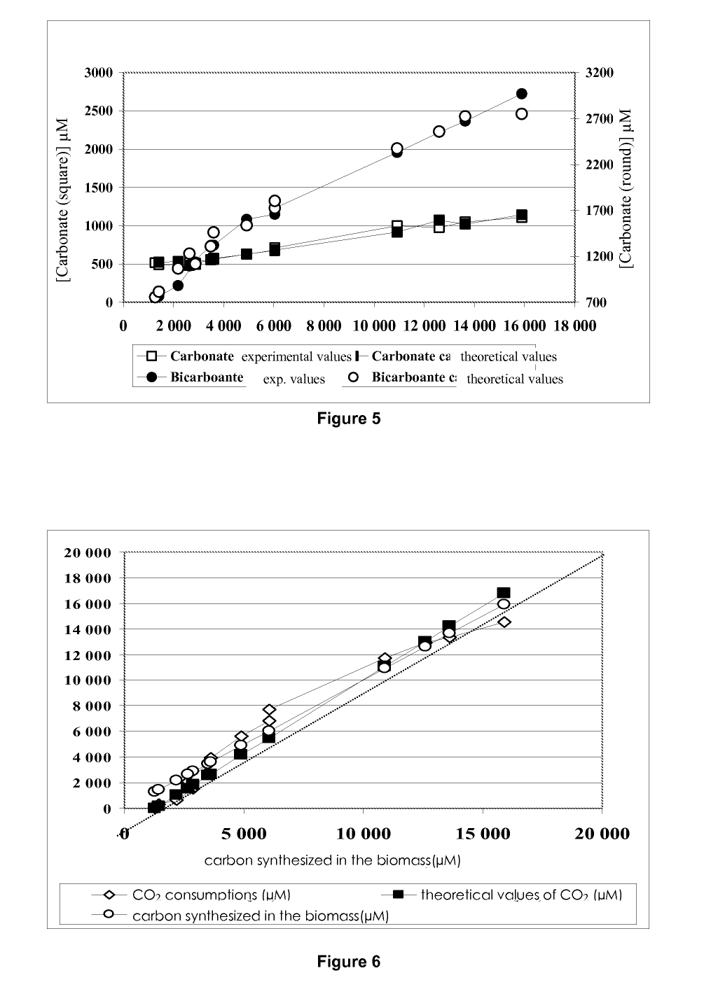

FIG. 5 shows the experimental values and the calculated values for hydrogen carbonate and carbonate concentrations as a function of the synthesized biomass C.

FIG. 6 shows the experimental values and the calculated values of injected CO₂ as a function of the synthesized biomass C.

For example, for a biomass concentration increase from 1 to 101 μM in the reactor, the amount of injected CO₂ will be 113.7 μmoles/l culture medium. These 13.7 μmoles/l culture medium represent the CO injected (113.7-100) in the carbonate system (distributed among the hydrogen carbonate and the carbonate) after incorporation of the nitrogen in the biomass, provided that the nitrogen is supplied in nitrate form (NO₃₋).

If the nitrogen source is provided in ammonium form, the opposite is observed as regards the pH evolution with acidification by H+ (1 H+ for 1 mole N incorporated in the biomass). The system cannot operate on a long-term basis for a biomass production according to this procedure. The respective concentrations of the two species are then rapidly decreased and the system is no longer functional. This situation is not favourable for optimum implementation of the method, and nitrogen is thus preferably supplied in nitrate form. Besides, it may be of interest to supply the nitrogen in form of calcium nitrate, thus allowing the calcium concentrations in the medium to be raised.

It is thus possible, from the initial hydrogen carbonate, carbonate and biomass concentrations, and knowing the biomass doubling rate (or the hydrogen carbonate consumption rate), to predict the respective concentration increase of the two species carbonate and hydrogen carbonate, associated with the biomass concentration increase and the CO₂ and nitrate consumption.

Indeed, it is important to be able to calculate the respective concentration increase of the two species carbonate and hydrogen carbonate.

Implementation of the Method According to the Invention: Growing Cyanobacteria on a Carbonate System with pH Regulated through CO₂ Injection. Culture Conditions in an Aqueous Medium in the Presence of Calcium

As described above, calcium carbonate precipitation is easily achieved batchwise by the cyanobacteria, provided that the carbonate and calcium concentrations are high enough to allow precipitation.

On the other hand, calcium carbonate precipitation cannot be done batchwise when using sea water with a composition of 1818 μM hydrogen carbonate, 272 μM carbonate and 10300 μM calcium, without adding inorganic carbon (CO₂ or HCO₃₋).

For the first precipitation event to occur, the calcium or the carbonate (from hydrogen carbonate) concentration has to be increased.

In order to solve this problem, the method according to the invention aims to modify the operating mode of the cyanobacteria culture method in order to produce calcium carbonate. In the method according to the invention, cyanobacteria are grown on a carbonate-containing aqueous medium (sea water or fresh water), in the presence of calcium, the pH being controlled by CO₂ injection. This culture method allows to produce carbonate ions by photosynthesis from the hydrogen carbonate ions, in order to obtain both a valorizable cyanobacterial biomass and a calcium carbonate (CaCO₃) precipitation by reaction of the carbonate in the presence of calcium.

In the case of a marine medium, it is also possible to introduce nitrogen in form of sea water supplemented in NaNO₃, without diluting and thus without decreasing the carbonate and calcium concentration values, which are important values for providing growth and production of calcium carbonate by means of this control procedure.

Furthermore, the calcium concentration increase has to be assessed by means of the calcium supply in calcium nitrate form.

Growth of the cyanobacterial biomass is first carried out on the hydrogen carbonate without pH regulation. Under such conditions, the respective hydrogen carbonate and carbonate concentrations change (basification through hydrogen carbonate consumption and carbonate production). For example, in the case of a pH value regulated to 9.5, with a concentration ratio between the two species of about 1/1, a concentration of the order of 780 μM is obtained for each species when starting the regulation. The biomass production (biomass C) is of the order of 515 μM. The mineral carbon consumption for biomass and calcium carbonate production occurs then through CO₂ injection. Under such conditions, the respective concentrations of these two species (hydrogen carbonate and carbonate) increase progressively as a function of the biomass concentration in the reactor at the beginning of the regulation and of the doubling time. The calculation procedures are those presented in paragraph “Cyanobacteria culture on a carbonate system with regulated pH and CO₂ injection—Culture conditions in a calcium-free medium”. As soon as the carbonate concentration reaches a value of 1100 μM, a first precipitation event occurs.

As mentioned above, CaCO₃ precipitation cannot be achieved in the presence of a final carbonate concentration of 1181 μM if a batchwise hydrogen carbonate consumption is used from sea water.

On the other hand, with the same carbonate concentration, CaCO₃ precipitation can be achieved in the carbonate system procedure with regulation through CO₂ injection (method according to the invention). In fact, when the pH regulation is initiated, the amount (or the concentration) of biomass present (around 515 μM biomass C) increases further during the time when the carbonate concentration shifts from 780 μM to 1100 μM. The biomass concentration increase for this value of 1100 μM carbonate can be calculated using the aforementioned equations.

The carbonate concentration drops to 300 μM after the first precipitation event; the pH is thus below the regulation set value due to the sequestration of part of the carbonate ions. Under such conditions, the pH value is no longer regulated. The hydrogen carbonate is then consumed only on the amount of hydrogen carbonate present in the medium, which has also increased until the precipitation event, and whose concentration can be calculated with the same equations as above. The pH regulation is started again for a hydrogen carbonate concentration that is again equal to that of the carbonate, i.e. of the order of 530 μM considering the distribution of the carbon ex hydrogen carbonate consumed, distributed between 1 biomass C and 1 carbonate C. The mineral carbon is consumed as soon as the regulation through CO₂ injection is activated again. Under such conditions, the respective concentrations of these two species are going to increase again progressively, as a function of the biomass concentration in the reactor and of the doubling time of this biomass. The calculation procedures are those presented in paragraph “Cyanobacteria culture on a carbonate system with regulated pH and CO₂ injection—Culture conditions in a calcium-free medium”. As soon as the carbonate concentration reaches a value of 1100 μM, a second precipitation event occurs.

This procedure can be repeated as many times as the calcium concentration allows, i.e. up to calcium exhaustion.

Valorization of the Biomass Obtained with the Method According to the Invention

Direct Valorization

The biomass obtained can undergo direct valorization. Due to its nitrogen content, the cyanobacterial biomass obtained with the method according to the invention can be used directly as a fertilizer.

Indirect Valorization

As explained above, the cyanobacterial biomass is probably not suited for valorization in form of fatty acid methyl esters.

The presence of sugar stocks can allow the biomass to be valorized after extraction thereof and conversion to sugars through alcoholic fermentation.

Recovery of this biomass and thermal treatment thereof by pyrolysis can also allow to obtain an oil making up a primary product for gasification with a Fischer-Tropsch synthesis.

These three approaches require separation of the biomass from the water of the culture medium. The biomass separated from the water can thus be used for energy valorization through thermal treatment.

This thermal valorization can be carried out by direct combustion and heat recovery, or by pyrolysis to generate an oil. The oil produced in this case can be used as a primary product for chemical syntheses for example, or valorized as a thermal energy source or in combustion engines, or as a primary product for gasification.

Biomass Methanogenesis

Separating the biomass from the water of the culture medium consumes energy and thus is costly. The biomass is therefore preferably treated using a biological anaerobic digestion process. Energy valorization of the biomass can thus be carried out by methanogenesis. Methanogenesis can be conducted in any suitable reactor of anaerobic digester type. In this preferred embodiment, anaerobic digestion is performed by biomass injection into an anaerobic digester during the nocturnal phase, when the culture medium has been made anaerobic by the respiration process in progress.

The biomass separated from the water (before and/or after anaerobic digestion) can be used for energy valorization through thermal treatment.

For a known elemental biomass composition (in terms of C, O, N, H, S), it is possible to predict the production of CO₂ and of methane. These calculations do not take account of the digestibility efficiencies that partly depend on the wall/membrane composition and on the protein content of the microorganism (methanogenesis inhibition is indeed possible due to high ammonium contents). The production of CH₄ and of CO₂ can be assessed by means of the formula as follows (that neither takes account of the requirements for the maintenance energy and the anabolism):

CₐH_{b}O_{c}N_{d}+(4a−b−2c+3d)/4H₂O→4(a+b−2c−3d)/8 CH₄+4(a−b+2c+3d)CO2+dNH₃

The composition of the cyanobacterial biomass obtained in a culture under CO₂ injection at a pH regulated to 9.5 and without nitrogen limitation is as follows (the elemental composition of the biomass is based on 100):

$$\begin{matrix}CONHS = & C & 45.9 \\ & H & 6.8 \\ & N & 8.9 \\ & O & 31.2 \\ & S & 0.6 \\ & Total & 93.4\end{matrix}$$

### EXAMPLE

Cyanobacteria are grown batchwise (closed medium, continuous culture) on a carbonate system with regulated pH and CO₂ injection in a marine medium, in sea water of initial composition 1818 μM hydrogen carbonate, 272 μM carbonate and 10300 μM calcium. The pH value is regulated around 9.5 to 10.

Balances were drawn up for a constant hydrogen carbonate consumption rate of 260 μM/hour (see Table 1 and Table 2).

TABLE 1
CO₂ consumption, biomass production, CaCO3 production, for each
phase with pH regulated by CO₂ injection (for a volume of 1 L marine medium)

| | number of | pH regulation | | | | |
|---|---|---|---|---|---|---|
| | pH | phase | HCO3- | CO2 | CaCO3 | Biomass |
| | regulation | duration | consumed | consumed | produced | produced |
| Process control | phases | (hours) | μM C | μM C | μM C | μM C |
| Batch on bicarbonate | | 1 | 1030 | | | 515 |
| Regulation phase | 1 | 37 | | 5300 | | 4680 |
| 1st precipitation | | | | | 800 | |
| Batch on bicarbonate | | 1 | 464 | | | 232 |
| Regulation phase | 2 | 58 | | 8540 | | 7573 |
| 2nd precipitation | | | | | 800 | |
| Batch on bicarbonate | | 1 | 464 | | | 232 |
| Regulation phase | 3 | 67 | | 9900 | | 8768 |
| 3rd precipitation | | | | | 800 | |
| Batch on bicarbonate | | 1 | 0 | | | 500 |
| 4th precipitation | | | | | 800 | |

A precipitation phenomenon takes place between each phase

TABLE 2
Cumulative CO₂ consumption, cumulative biomass production, CaCO3
production, for each phase with pH regulated by CO₂ injection
(for a volume of 1 L marine medium)

| | | Cumulative | | | | |
|---|---|---|---|---|---|---|
| | Number of | pH | | | | |
| | pH | regulation | HCO3- | CO2 | CaCO3 | Biomass |
| | regulation | phase | consumed | consumed | produced | produced |
| Process control | phases | (hours) | μM C | μM C | μM C | μM C |
| Batch on bicarbonate | | 1 | 1030 | | | 515 |
| Regulation phase | 1 | 38 | | 5300 | | 5195 |
| 1st precipitation | | | | | 800 | |
| Batch on bicarbonate | | 39 | 1494 | | | 5427 |
| Regulation phase | 2 | 97 | | 13840 | | 13000 |
| 2nd precipitation | | | | | 1600 | |
| Batch on bicarbonate | | 98 | 1958 | | | 13232 |
| Regulation phase | 3 | 165 | | 23740 | | 22000 |
| 3rd precipitation | | | | | 2400 | |
| Batch on bicarbonate | | 166 | 0 | | | 22500 |
| 4th precipitation | | | | | 3200 | |

A precipitation phenomenon takes place between each phase.

The hydrogen carbonate and carbonate concentrations at the end of each precipitation stage are of the order of 1000 μM and 300 μM respectively.

On the basis of this hydrogen carbonate consumption rate of 260 μM/hour, this cyanobacteria culture procedure in the presence of a sea water medium with CO₂ injection allows the following balance to be obtained for 1 liter marine medium:

Consumption of 1818 μM hydrogen carbonate and 23740 μM CO₂,

Production of 22500 μM biomass C and 3200 μM CaCO₃ C.

This hydrogen carbonate consumption rate leads to a final biomass concentration of 588 mg/L, an OD (absorbance at 600 nm) of 2.8 and a cell density (number of cells/ml) of the order of 9.4 10⁸ cells/ml.

The process time is approximately 1 week.

The production (concentration) of biomass of 22500 μM C requires a cumulative nitrate concentration (preferably calcium nitrate) of 22500*0.165=3712 μM, i.e. 3.7 mM that add up to the 10.3 mM calcium of the sea water. This situation can only be favourable to the appearance of the precipitation event.

The annualized balance for a facility volume of 5000 m³, i.e. one hectare with 50 cm water depth, is:

Sea water volume used 5000 m³

CO₂ (gaseous) consumption 260 T CO₂

Biomass production 147 T biomass (dry weight)

CaCO₃ production 81 T CaCO₃ (dry weight).

This hydrogen carbonate consumption rate leads to a final biomass concentration of 588 mg/L, an OD of 2.8 and a population of the order of 9.4 10⁸.

It may be necessary to reassess the hydrogen carbonate consumption rates in this operating mode, under conditions of non-limitation of elements (micro or macro). Apart from this problem of limitation by nutrients, photolimitation can contribute to decreasing the progression of the biomass concentration increase towards an asymptote. Independently of this problem of growth rate limitation by nutrients, photolimitation tends to limit the maximum biomass concentration with which a production cycle can be ended (over one week). In an operating mode, this concentration value should not be exceeded.

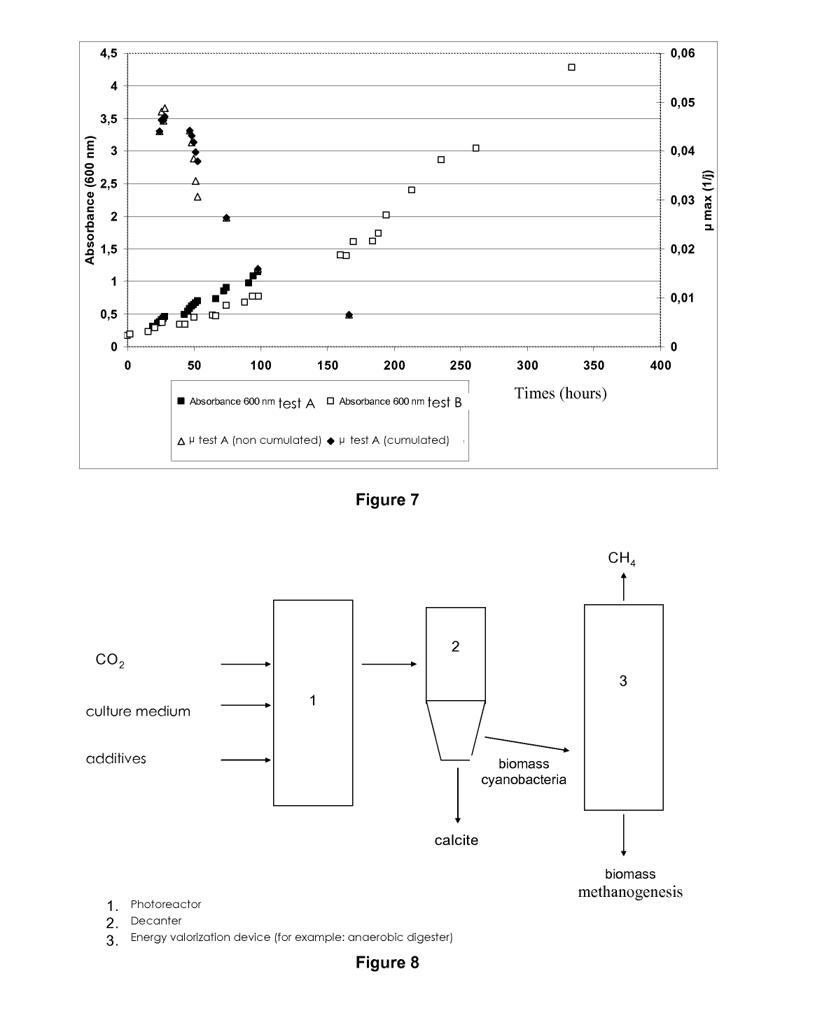

The data obtained on the one hand for test A [FIG. 7: data of μ (squares and triangles) and OD 660 nm (diamonds)] for which the maximum OD values are 1.4 on the one hand and the data obtained for test B [FIG. 6: OD 660 nm (black-edged squares)] for which the maximum OD values are 4.3 show that the growth progression does not occur with μ being constant.

The current data relative to algae biomass production in ponds concern productivities of the order of 40 T/hectare (dry weight), with eukaryotic algae.

The cyanobacteria biomass is probably not suited for valorization in form of fatty acid methyl esters.

The presence of sugar stocks could allow the biomass to be valorized after extraction thereof and conversion to sugars through alcoholic fermentation.

Recovery of this biomass and thermal treatment thereof by pyrolysis could allow to obtain an oil that would be a primary product for gasification with a Fischer-Tropsch synthesis.

These three approaches however require separating the biomass from the water of the culture medium. Such a separation stage consumes energy and is thus costly. Treating the biomass by means of a biological anaerobic digestion process is therefore suggested according to the diagram of FIG. 8, where reference number 1 is a photoreactor, reference number 2 a decanter and reference number 3 an anaerobic digester, for example.

1) An integrated CO₂ biological capture method using cyanobacteria allowing production of biomass and of calcite (CaCO₃) and comprising the following stages:
a) growing cyanobacteria in the presence of calcium in an aqueous medium comprising carbonate ions and hydrogen carbonate ions ;
b) achieving photosynthesis by the cyanobacteria on the hydrogen carbonate ions so as to produce a cyanobacterial biomass consisting of the elements carbon, oxygen, nitrogen, hydrogen and sulfur, and to cause precipitation of calcium carbonate (CaCO₃) ;
c) supplying inorganic carbon by injecting CO₂ in order to regulate the pH during the photosynthesis reaction.

2) A method as claimed in claim 1, wherein the CO₂ is atmospheric and the cyanobacteria are grown in at least one open reactor.

3) A method as claimed in claim 1, wherein at least part of the CO₂ injected comes from industrial fumes/discharges.

4) A method as claimed in claim 1, wherein stages a), b), c) are repeated until exhaustion of the calcium in the medium.

5) A method as claimed in claim 1, wherein the pH is regulated to a value ranging between 9 and 10.

6) A method as claimed in claim 1, wherein the culture medium is a synthetic aqueous medium containing calcium and a carbonate system, or a marine medium.

7) A method as claimed in claim 1, wherein nitrogen is supplied to the medium in form of nitrate ions.

8) A method as claimed in claim 7, wherein the nitrate ions are calcium nitrate.

9) A method as claimed in claim 1, wherein cyanobacteria are grown under continuous or semi-continuous conditions, or batchwise.

10) A method as claimed in claim 1, wherein a stage of anaerobic digestion of the cyanobacterial biomass is carried out in order to convert the biomass to methane.

11) A method as claimed in claim 10, wherein the anaerobic digestion stage is carried out by injecting the biomass into an anaerobic digester during the nocturnal phase.

12) A method as claimed in claims 10, wherein the biomass anaerobic digestion stage is carried out directly after the production stage.

13) A method as claimed in claim 1, wherein the biomass is separated from the medium water for thermal valorization purposes.

14) A method as claimed in claim 13, wherein the biomass is valorized by direct combustion and heat recovery, or by pyrolysis in order to generate an oil.

15) A method as claimed in claim 1, wherein the biomass obtained is used as an energy source.

16) A method as claimed in claim 1, wherein the biomass obtained is used as a fertilizer.

17) A method as claimed in claim 1, used for calcite production (CaCO₃) and for CO₂ mineral sequestration.
